(19) United States
(12) Patent Application Publication
(10) Pub. No.: US 2020/0219610 A1
(43) Pub. Date: Jul. 9, 2020

(54) SYSTEM AND METHOD FOR PROVIDING PREDICTION MODELS FOR PREDICTING A HEALTH DETERMINANT CATEGORY CONTRIBUTION IN SAVINGS GENERATED BY A CLINICAL PROGRAM

(71) Applicant: KONINKLIJKE PHILIPS N.V., EINDHOVEN (NL)

(72) Inventors: Daniele DE MASSARI, Eindhoven (NL); Jorn OP DEN BUIJS, Eindhoven (NL)

(21) Appl. No.: 16/625,022

(22) PCT Filed: Jun. 28, 2018
(86) PCT No.: PCT/EP2018/067377

§ 371 Date: Dec. 20, 2019

Related U.S. Application Data
(60) Provisional application No. 62/528,582, filed on Jul. 5, 2017.

(51) Int. Cl. G16H 40/20; G16H 50/70; G06N 20/20; G16H 10/60; G16H 50/20

(52) U.S. Cl. CPC G16H 40/20; G16H 50/70; G16H 50/20; G16H 10/60; G06N 20/20

(57) ABSTRACT

The present disclosure pertains to a system for providing prediction models for predicting a health determinant category contribution in savings generated by a clinical program. In some embodiments, the system obtains healthcare data including (i) historical and financial data corresponding to one or more clinical programs, (ii) demographic, clinical, and behavioral data of one or more patients, and (iii) environmental factors associated with the one or more clinical programs; defines one or more health determinant categories; generates prediction models related to a contribution of one or more constituents of the one or more health determinant categories to savings generated by the one or more clinical programs; generates one or more predictions related to a contribution of the one or more health determinant categories to the savings generated by the one or more clinical programs; and effectuates, via a user interface, presentation of the one or more predictions.

### BACKGROUND

### 1. Field

The present disclosure pertains to a system and method for providing prediction models for predicting a health determinant category contribution in savings generated by a clinical program.

### 2. Description of the Related Art

A clinical program may be affected by one or more different health determinants and the outcomes of such programs may be spread within a population. As such, determination of the success of the clinical program may be affected by different contributions of one or more health determinants, some of which may not be healthcare related and hence not under a direct control of a healthcare provider. For example, the clinical program may be affected by schools, polluting industries, and/or other non-healthcare related determinants. Furthermore, with healthcare providers deploying multiple clinical programs targeting different populations and different diseases, it may be difficult to quantify how the different determinants contribute to the overall savings generated by the clinical programs. Although computer-assisted data analysis systems exist, such systems may be unable to evaluate individual contributions and/or impacts of each of the health determinants on a clinical program investment when the healthcare providers operate under bundled or capitation payment models. These and other drawbacks exist.

### SUMMARY

Accordingly, one or more aspects of the present disclosure relate to a system configured to provide prediction models for predicting a health determinant category contribution in clinical outcomes. The system comprises one or more processors configured by machine readable instructions and/or other components. The system is configured to: obtain healthcare data including (i) historical and financial data corresponding to one or more clinical programs, (ii) demographic, clinical, and behavioral data of one or more patients, and (iii) one or more environmental factors associated with the one or more clinical programs; define one or more health determinant categories; generate prediction models based on the healthcare data and the one or more health determinant categories such that at least one of the prediction models is configured to generate a prediction related to a contribution of one or more constituents of the one or more health determinant categories to savings generated by the one or more clinical programs; generate one or more predictions based on the prediction models, the predictions being related to a contribution of the one or more health determinant categories to the savings generated by the one or more clinical programs; and effectuate, via a user interface, presentation of the one or more predictions.

Another aspect of the present disclosure relates to a method for providing prediction models for predicting a health determinant category contribution in clinical outcomes with a system. The system comprises one or more processors configured by machine readable instructions and/or other components. The method comprises: obtaining, with the one or more processors, healthcare data including (i) historical and financial data corresponding to one or more clinical programs, (ii) demographic, clinical, and behavioral data of one or more patients, and (iii) one or more environmental factors associated with the one or more clinical programs; defining, with the one or more processors, one or more health determinant categories; generating, with the one or more processors, prediction models based on the healthcare data and the one or more health determinant categories such that at least one of the prediction models is configured to generate a prediction related to a contribution of one or more constituents of the one or more health determinant categories to savings generated by the one or more clinical programs; generating, with the one or more processors, one or more predictions based on the prediction models, the predictions being related to a contribution of the one or more health determinant categories to the savings generated by the one or more clinical programs; and effectuating, with a user interface, presentation of the one or more predictions.

Still another aspect of present disclosure relates to a system for providing prediction models for predicting a health determinant category contribution in clinical outcomes. The system comprises means for obtaining healthcare data including (i) historical and financial data corresponding to one or more clinical programs, (ii) demographic, clinical, and behavioral data of one or more patients, and (iii) one or more environmental factors associated with the one or more clinical programs; means for defining one or more health determinant categories; means for generating prediction models based on the healthcare data and the one or more health determinant categories such that at least one of the prediction models is configured to generate a prediction related to a contribution of one or more constituents of the one or more health determinant categories to savings generated by the one or more clinical programs; means for generating one or more predictions based on the prediction models, the predictions being related to a contribution of the one or more health determinant categories to the savings generated by the one or more clinical programs; and means for effectuating presentation of the one or more predictions.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 illustrates a system configured to provide prediction models for predicting a health determinant category contribution in clinical outcomes, in accordance with one or more embodiments.

FIG. 2 illustrates geospatially merged patient health determinant data and environmental determinant data, in accordance with one or more embodiments.

FIG. 3 illustrates contributions of individual health determinant constituents to patient cost savings, in accordance with one or more embodiments.

FIG. 4 illustrates determination of distributed savings, in accordance with one or more embodiments.

FIG. 5 illustrates interaction of different stakeholders in a healthcare system.

FIG. 6 illustrates a method for providing prediction models for predicting a health determinant category contribution in clinical outcomes, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

With the rise in the general population's age, the incidence rate of chronic diseases and healthcare costs may be increasing in many countries. As such, healthcare systems may require new and more efficient methods to deliver care to patients. These methods may include the deployment of integrated care programs wherein a multi-disciplinary team coordinates the delivery of care to patients who suffer from more than one condition. The care management of complex patients reduces redundancies, improves patients' experience, provides a unique point of contact with the healthcare system and allows for standardized care delivery. In case of acute events, such as strokes, myocardial infarctions, orthopedic surgeries, and/or other acute events which may require subsequent rehabilitation periods (e.g. 1-month, 3-months, etc.) other programs may be rolled out. Such methods may further aim to reduce or, in most of the cases, maintain the costs of delivering care constant from year to year. With the adoption of capitation or bundled payment models healthcare providers may be able to generate savings if they are able to spend less than the amount received from the payers while achieving the target clinical outcomes.

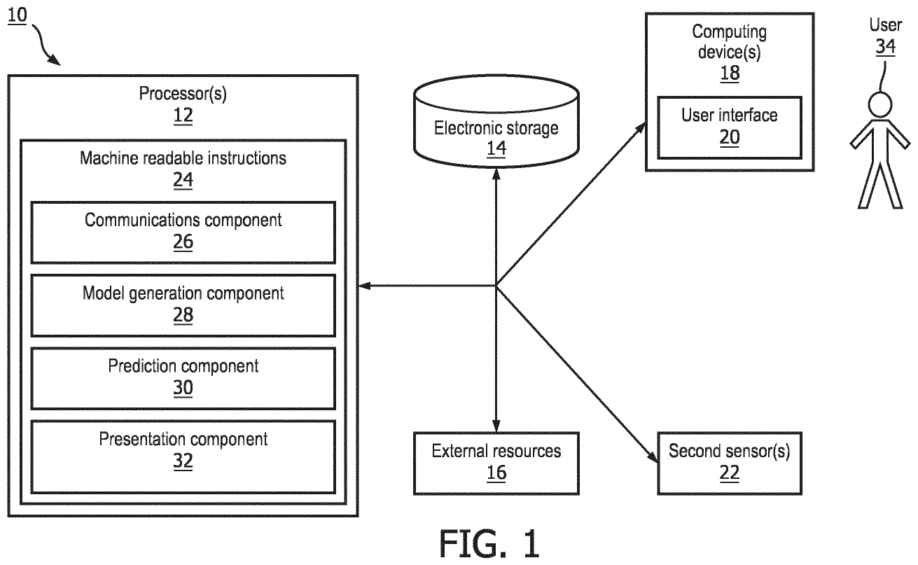

FIG. 1 is a schematic illustration of a system 10 configured to provide prediction models for predicting a health determinant category contribution in clinical outcomes. In some embodiments, system 10 is configured to generate a prediction model (e.g., statistical model, machine learning algorithm, etc.) which generates an estimation of the contributions that different determinants had on the savings generated by a clinical program. In some embodiments, the different determinants include one or more of a social determinant, a physical environmental determinant, a healthcare determinant, a genetic determinant, a behavioral determinant, a biological determinant, and/or other determinants affecting the success of a clinical program, intervention, therapy, and/or other programs. In some embodiments, system 10 is configured to obtain healthcare data, define one or more health determinant categories, extract features from the healthcare data and group the extracted data according to the categories of health determinants, generate prediction models related to a contribution of individual constituents corresponding to each of the health determinant categories, generate a prediction related to a contribution of one or more health determinant categories to the savings generated by the clinical program, and effectuate presentation of the prediction.

In some embodiments, system 10 comprises one or more processors 12, electronic storage 14, external resources 16, computing device 18, one or more sensors 22, or other components.

Electronic storage 14 comprises electronic storage media that electronically stores information (e.g., criteria, mathematical equations, predictions, etc.). The electronic storage media of electronic storage 14 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., computing device 18, processor 12, etc.). In some embodiments, electronic storage 14 may be located in a server together with processor 12, in a server that is part of external resources 16, in a computing device 18, and/or in other locations. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 14 may store software algorithms, information determined by processor 12, information received via computing devices 18 and/or graphical user interface 20 and/or other external computing systems, information received from external resources 16, and/or other information that enables system 10 to function as described herein.

External resources 16 include sources of information and/or other resources. For example, external resources 16 may include data related to the financial information of one or more clinical programs, demographic, clinical, and behavioral data of a patient population, environmental factors, and/or other information. In some embodiments, external resources 16 include sources of information such as databases, websites, etc., external entities participating with system 10 (e.g., a medical records system of a health care provider that stores medical history information for populations of patients), one or more servers outside of system 10, and/or other sources of information. In some embodiments, external resources 16 include components that facilitate communication of information such as a network (e.g., the internet), electronic storage, equipment related to Wi-Fi technology, equipment related to Bluetooth® technology, data entry devices, sensors, scanners, and/or other resources. External resources 16 may be configured to communicate with processor 12, computing device 18, electronic storage 14, and/or other components of system 10 via wired and/or wireless connections, via a network (e.g., a local area network and/or the internet), via cellular technology, via Wi-Fi technology, and/or via other resources. In some embodiments, some or all of the functionality attributed herein to external resources 16 may be provided by resources included in system 10.

Computing devices 18 are configured to provide an interface between user 34 (e.g., a healthcare organization representative, an accountable care organization representative, a payer, a clinical program stakeholder, an investor, etc.), and/or other users, and system 10. In some embodiments, individual computing devices 18 are and/or are included in desktop computers, laptop computers, tablet computers, smartphones, and/or other computing devices associated with individual caregivers 14, individual patients 12, and/or other users. In some embodiments, individual computing devices 18 are, and/or are included in equipment used in insurer's offices, hospitals, doctor's offices, and/or other facilities. Computing devices 18 are configured to provide information to and/or receive information from user 34, and/or other users. For example, computing devices 18 are configured to present a graphical user interface 20 to user 34 to facilitate entry and/or selection of a descriptive statistic and a margin of error (e.g., as described below). In some embodiments, graphical user interface 20 includes a plurality of separate interfaces associated with computing devices 18, processor 12, and/or other components of system 10; multiple views and/or fields configured to convey information to and/or receive information from user 34, and/or other users; and/or other interfaces.

In some embodiments, computing devices 18 are configured to provide user interface 20, processing capabilities, databases, or electronic storage to system 10. As such, computing devices 18 may include processor 12, electronic storage 14, external resources 16, or other components of system 10. In some embodiments, computing devices 18 are connected to a network (e.g., the internet). In some embodiments, computing devices 18 do not include processor 12, electronic storage 14, external resources 16, or other components of system 10, but instead communicate with these components via the network. The connection to the network may be wireless or wired. For example, processor 12 may be located in a remote server and may wirelessly cause presentation of the one or more predictions via the user interface to a care provider on computing devices 18 associated with that caregiver (e.g., a doctor, a nurse, a central caregiver coordinator, etc.).

Examples of interface devices suitable for inclusion in user interface 20 include a camera, a touch screen, a keypad, touch sensitive or physical buttons, switches, a keyboard, knobs, levers, a display, speakers, a microphone, an indicator light, an audible alarm, a printer, tactile haptic feedback device, or other interface devices. The present disclosure also contemplates that computing devices 18 includes a removable storage interface. In this example, information may be loaded into computing devices 18 from removable storage (e.g., a smart card, a flash drive, a removable disk, etc.) that enables caregivers or other users to customize the implementation of computing device 18. Other exemplary input devices and techniques adapted for use with Computing devices 18 or the user interface include an RS-232 port, RF link, an IR link, a modem (telephone, cable, etc.), or other devices or techniques.

One or more sensors 22 are configured to generate output signals conveying information related to geographical areas where the one or more patients spend their time. In some embodiments, one or more sensors 22 include a GPS and/or other sensors. In some embodiments, one or more sensors 22 are embedded in the one or more users' mobile device (e.g., cell phone), wearable device (e.g., smart watch), and/or other devices.

Processor 12 is configured to provide information processing capabilities in system 10. As such, processor 12 may comprise one or more of a digital processor, an analog processor, a digital circuit designed to process information, an analog circuit designed to process information, a state machine, or other mechanisms for electronically processing information. Although processor 12 is shown in FIG. 1 as a single entity, this is for illustrative purposes only. In some embodiments, processor 12 may comprise a plurality of processing units. These processing units may be physically located within the same device (e.g., a server), or processor 12 may represent processing functionality of a plurality of devices operating in coordination (e.g., one or more servers, computing device 18, devices that are part of external resources 16, electronic storage 14, or other devices.)

In some embodiments, processor 12, external resources 16, computing devices 18, electronic storage 14, and/or other components may be operatively linked via one or more electronic communication links. For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 12 is configured to communicate with external resources 16, computing devices 18, electronic storage 14, and/or other components according to a client/server architecture, a peer-to-peer architecture, and/or other architectures.

As shown in FIG. 1, processor 12 is configured via machine-readable instructions 24 to execute one or more computer program components. The computer program components may comprise one or more of a communications component 26, a model generation component 28, a prediction component 30, a presentation component 32, or other components. Processor 12 may be configured to execute components 26, 28, 30, or 32 by software; hardware; firmware; some combination of software, hardware, or firmware; or other mechanisms for configuring processing capabilities on processor 12.

It should be appreciated that although components 26, 28, 30, and 32 are illustrated in FIG. 1 as being co-located within a single processing unit, in embodiments in which processor 12 comprises multiple processing units, one or more of components 26, 28, 30, or 32 may be located remotely from the other components. The description of the functionality provided by the different components 26, 28, 30, or 32 described below is for illustrative purposes, and is not intended to be limiting, as any of components 26, 28, 30, or 32 may provide more or less functionality than is described. For example, one or more of components 26, 28, 30, or 32 may be eliminated, and some or all of its functionality may be provided by other components 26, 28, 30, or 32. As another example, processor 12 may be configured to execute one or more additional components that may perform some or all of the functionality attributed below to one of components 26, 28, 30, or 32.

Communications component 26 is configured to obtain healthcare data including (i) historical and financial data corresponding to one or more clinical programs, (ii) demographic, clinical, and behavioral data of one or more patients, (iii) one or more environmental factors associated with the one or more clinical programs, and/or other data. In some embodiments, communications component 26 obtains the healthcare data from electronic storage 14 (e.g., medical and financial data saved on electronic storage 14), external resources 16 (e.g., healthcare organization electronic records), via one or more surveys and/or queries, and/or via other resources.

In some embodiments, communications component 26 is configured to define one or more health determinant categories. In some embodiments, the one or more health determinant categories includes one or more of patients' behavioral information, patients' clinical and demographic information, healthcare providers' information, environmental information, pre-post program behavioral information, pre-post program clinical information, pre-post program healthcare provider information, pre-post environmental information, and/or other categories.

In some embodiments, the patients' behavioral information comprises one or more of a score associated with the one or more patients' activation, a number of disenrollments from previous clinical programs, a number of previous successful clinical programs, a number of scheduled healthcare appointments attended, a number of scheduled healthcare appointments missed, a psychological profiling associated with the one or more patients, and/or other information.

In some embodiments, the patients' clinical and demographic information comprises one or more of an age, a gender, a primary diagnosis, a time since primary diagnosis, a number of secondary diagnosis, a frailty index, a 30-days readmissions risk score, one or more lab test results, a weight, a body mass index, and/or other information.

In some embodiments, the healthcare providers' information comprises one or more of a number of medical doctors involved with the one or more clinical programs, a number of nurses involved with the one or more clinical programs, a total number of healthcare professionals involved with the one or more clinical programs, a number of available ambulatories, a number of hospitals, a mean minimal distance between each ambulatory and hospital and the one or more patients' home, a number of clinical program runs per year, a number of pharmacies, and/or other information. In some embodiments, the healthcare providers' information includes (i) a capitated payment received by a healthcare provider per patient and (ii) actual costs incurred by the healthcare provider to care for individual ones of the one or more patients.

In some embodiments, the environmental information comprises one or more of acres of green parks, a number of fast food restaurants, a number of community groups, a number of private schools in the region inhabited by the one or more patients, a number of public schools in the region inhabited by the one or more patients, and/or other information.

In some embodiments, the pre-post program behavioral information comprises a difference between the patients' behavioral information before and after the one or more clinical programs and/or other information. In one use case, for example, the patients' behavioral information may include a patient's activation score. The pre-program behavior information may indicate the patient does not have the necessary skills, knowledge, and/or motivation to manage their diet. The post-program behavior information may indicate that the patient is conscious of his/her dietary restrictions.

In some embodiments, the pre-post program clinical information comprises a difference between the patients' clinical and demographic information before and after the one or more clinical programs and/or other information. In one use case, for example, the clinical information comprises a patient's body mass index, weight, lab values, and/or other information. The pre-program clinical information may indicate that the patient is overweight and has high cholesterol. The post-program clinical information may indicate that the patient's body weight and cholesterol has decreased.

In some embodiments, the pre-post program healthcare provider information comprises a difference between the healthcare providers' information before and after the one or more clinical programs and/or other information. In one use case, for example, the healthcare provider information comprises a number of hospitals. The pre-program clinical information may be indicative of one hospital within a predetermined radius. The post-program clinical information may be indicative of three hospitals within the predetermined radius.

In some embodiments, the pre-post environmental information comprises a difference between the environmental information before and after the one or more clinical programs and/or other information. In one use case, for example, the environmental information comprises acres of green parks. The pre-program environmental information may be indicative of approximately five acres of green park space in a particular geographical area. The post-program environmental information may be indicative of approximately 20 acres of green park space in the particular geographical area.

In some embodiments, communications component 26 is configured to receive the output signals generated by one or more sensors 22. In some embodiments, communications component 26 is configured to merge, based on the output signals, one or both of the patients' behavioral information or the patients' clinical and demographic information with one or both of the healthcare providers' information or the environmental information.

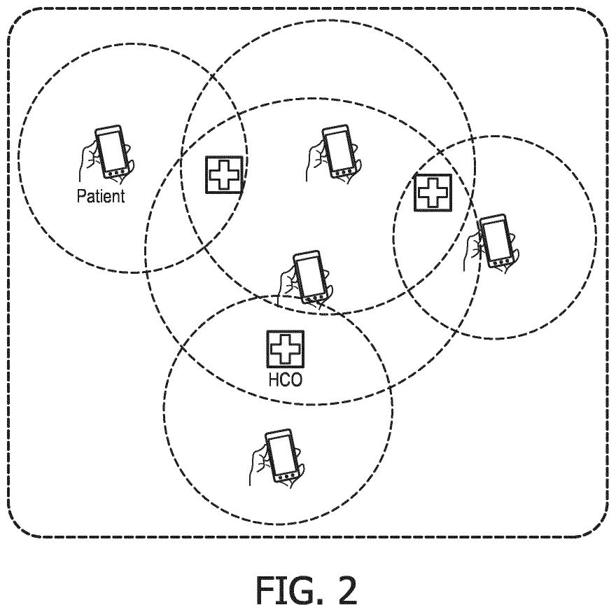

By way of a non-limiting example, FIG. 2 illustrates geospatially merged patient health determinant data and environmental determinant data, in accordance with one or more embodiments. As shown in FIG. 2, patient health determinant data (e.g. behavioral, demographics, etc.) are merged with environmental determinant data based on the geospatial coordinates as logged via one or more sensors 22 (e.g., the patients' mobile device). In some embodiments, geospatial coordinates are utilized to (i) localize the whereabouts of the patients and (ii) retrieve, from publicly available databases, information related to the an environment (e.g. number and sizes of parks, number and type of industries, etc.) where the patients are located. As such, communications component 26 facilitates detection of the geographical area where the one or more patients spend their time. For example, in case of long commuting time, business trips, temporary relocation, holidays, etc. the one or more patients may be in the reach of completely different subsets of recipient healthcare organizations (HCO). In some embodiments, communications component 26 is configured to provide the merged and aggregated data to one or more recipients (e.g., healthcare organizations) within a predetermined radial distance of the one or more patients.

Returning to FIG. 1, model generation component 28 is configured to generate prediction models based on the healthcare data and the one or more health determinant categories such that at least one of the prediction models is configured to generate a prediction related to a contribution of one or more constituents of the one or more health determinant categories to savings generated by the one or more clinical programs. In some embodiments, model generation component 28 is configured to determine patient cost savings by determining a difference between the capitated payment received by the healthcare provider per patient and the actual incurred costs by the healthcare provider to care for the individual ones of the one or more patients. In some embodiments, model generation component 28 is configured to determine population cost savings by determining a sum of the patient cost savings for all of the patients involved with the one or more clinical programs.

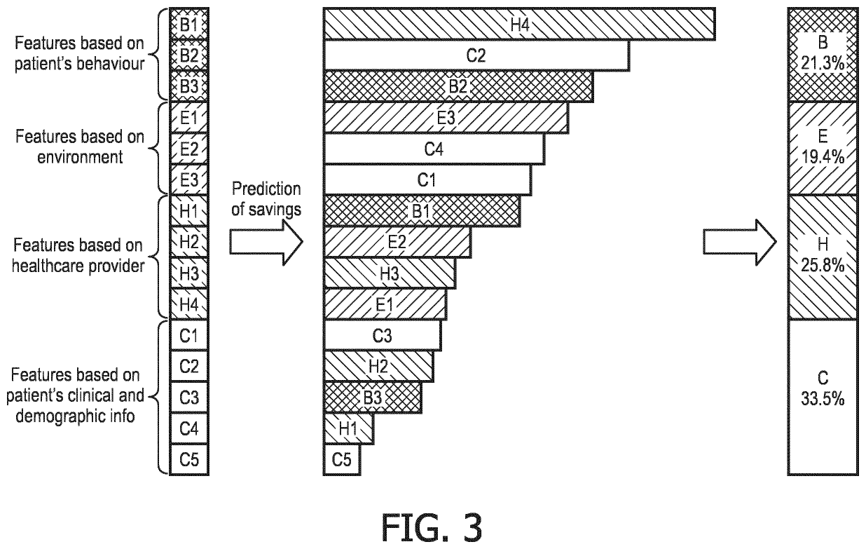

In some embodiments, model generation component 28 is configured such that the contribution of one or more constituents of the one or more health determinant categories to the savings generated by the one or more clinical programs is predicted based on a Random forests model and/or other models. For example, model generation component 28 may (i) generate a feature vector, based on one or more of the healthcare data, the merged data, health determinant categories, and/or other data provided by communications component, per patient and (ii) aggregate the feature vectors into a feature matrix having a number of rows equal to the number of patients in a target population of the one or more clinical programs. In some embodiments, model generation component 28 is configured to determine a regression between the feature matrix and the determined patient cost savings. By way of a non-limiting example, FIG. 3 illustrates contributions of individual health determinant constituents to patient cost savings, in accordance with one or more embodiments. As shown in FIG. 3, contribution of individual constituents (e.g., B1, B2, and B3 are constituents of the patients' behavioral information determinant category, E1, E2, and E3 are constituents of the environmental information determinant category, H1, H2, H3, and H4 are constituents of the healthcare providers' information determinant category, and C1, C2, C3, C4, and C5 are constituents of the patients' clinical and demographic information determinant category) are shown according to their corresponding rank (see, e.g., central bar plot).

In some embodiments, model generation component 28 is configured to determine, based on one or more of the pre-post program behavioral information, pre-post program clinical information, pre-post program healthcare provider information, or pre-post environmental information, the contribution of a change in one or more of the patients' behavioral information, the patients' clinical and demographic information, the healthcare providers' information, or the environmental information to the savings generated by the one or more clinical programs.

In some embodiments, model generation component 28 is configured such that, with respect to a determined contribution of one or more health determinant categories, at least one of the prediction models is configured to generate a prediction related to a potential future contribution of the one or more health determinant categories within a given time window. For example, model generation component 28 may determine (i) a contribution of the one or more health determinant categories to the savings generated by the clinical program immediately subsequent to the completion of the clinical program and (ii) potential contribution of the one or more health determinant categories to the savings accrued by the clinical program within one year from the date of completion of the clinical program (e.g., schools, polluting industries, and/or other determinants, since the contribution of one or more health determinant categories may not be available immediately subsequent to the completion of the clinical program).

In some embodiments, the one or more prediction models may be and/or include a neutral network that is trained and utilized for generating predictions (described below). As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neutral unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

In some embodiments, model generation component 28 is configured to update the prediction models based on (i) newly obtained healthcare data, (ii) data obtained from different programs offered by different service providers which target the same patient population, or (iii) other information. In some embodiments, model generation component 28 is configured to automatically update the prediction models responsive to updated contribution values, and/or other information being obtained. For example, the prediction models may be updated such that values corresponding to estimated contribution values of the one or more health determinant categories determined immediately subsequent to completion of a clinical program are replaced with values corresponding to updated contribution values obtained two years following the completion of the clinical program.

Returning to FIG. 1, prediction component 30 is configured to generate one or more predictions based on the prediction models. In some embodiments, the predictions are related to a contribution of the one or more health determinant categories to the savings generated by the one or more clinical programs. In some embodiments, prediction component 30 is configured to determine a sum of the contributions of all of the constituents of individual ones of the health determinant categories to predict the contribution of each health determinant category to the savings generated by the one or more clinical programs. In some embodiments, prediction component 30 is configured to determine weights per category as a percentage in which each health determinant category represents in the overall sum of contributions. By way of a non-limiting example, FIG. 3 illustrates the relative weights of each health determinant category (see, e.g., right side of the graph). As shown in FIG. 3, the patients' clinical and demographic information determinant is indicated as contributing the most (e.g., 33.5%) and the environmental information determinant is indicated as having the least contribution (e.g., 19.4%) to the overall success and/or cost of the clinical program.

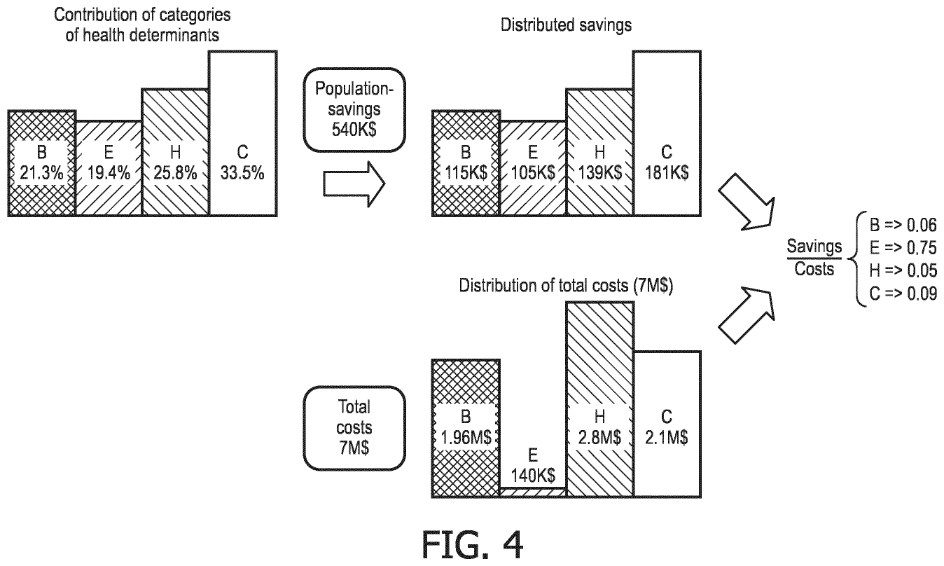

In some embodiments, the weights are indicative of an estimation of the contributions that each health determinants has into the overall savings generated by the one or more clinical programs. In some embodiments, prediction component 30 is configured to determine a distribution of the population cost savings across the different categories of health determinants using the category weights. By way of a non-limiting example, FIG. 4 illustrates determination of distributed savings, in accordance with one or more embodiments. As shown in FIG. 4, distributed savings are determined based on the estimated contribution of each category of health determinant. In FIG. 4, the actual costs incurred by the healthcare provider are divided into the different categories of health determinants based on the details of the clinical program. For example, responsive to a clinical program comprising a specific training to empower and activate the patients (e.g., to have an impact on the patient's behavior), all the costs incurred to run such training are attributed to the “Patient's behavior” category. As another example, if the program entails the renting of a facility or additional training for the involved healthcare professionals or the purchase of specific devices, all those costs are attributed to the “healthcare provider” category.

In some embodiments, prediction component 30 is configured to (i) determine a ratio between the determined cost savings and the actual incurred costs per the one or more health determinant categories and (ii) identify the most cost-effective health determinant category based on the determined ratio. For example, as shown in FIG. 4, the ratio of savings to costs has been determined for each of the categories. In this example, environmental features are indicated as having the highest ratio value (return on investment). Therefore, among all of the contributing health determinant categories for this clinical program, the expenses incurred for environmental features category may be the most cost-effective.

In some embodiments, prediction component 30 is configured to facilitate determination of profitability of an investment in each category of health determinant for a clinical program based on the determined weights, the determined ratios, and/or other factors.

Returning to FIG. 1, presentation component 32 is configured to effectuate, via user interface 20, presentation of the one or more predictions. Referring to FIG. 4, presentation component 32 is configured to effectuate presentation of one or more of the contribution of categories of health determinants to the one or more clinical programs, the determined distributed savings, distribution of total costs, the determined ratio between attributed savings and actual costs per category of health determinants, and/or other information. In some embodiments, presentation component 32 is configured such that presentation of information is effectuated via one or more of a bar chart, pie chart, a histogram bar plot, a Pareto chart, a scatter plot, a categorical (e.g., health determinant) summary, an excel spreadsheet, and/or other presentation methods.

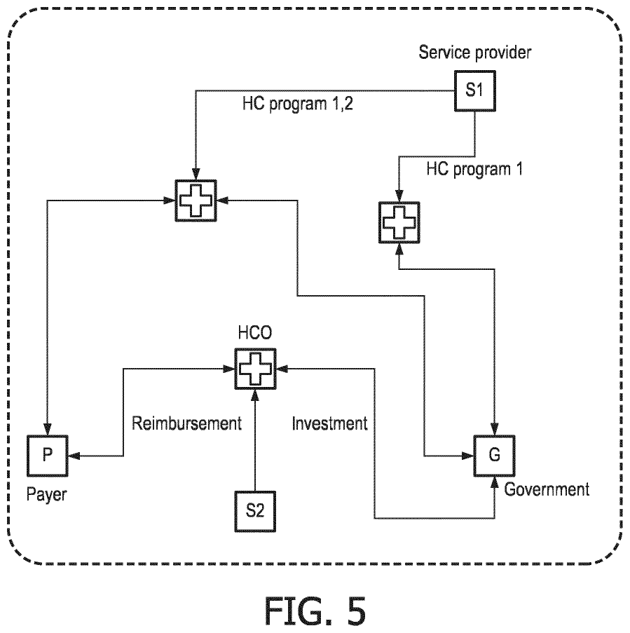

In some embodiments, presentation component 32 is configured to effectuate presentation of how different healthcare providers, healthcare organizations, payers, government institutes, and/or other stakeholders interact in a stakeholder map. By way of a non-limiting example, FIG. 5 illustrates interaction of different stakeholders in a healthcare system, in accordance with one or more embodiments. As shown in FIG. 5, service providers may be associated with multiple healthcare organizations for provision of health care programs (e.g., post-acute care and rehabilitation programs, telehealth and telecare programs). In some embodiments, presentation component 32 is configured to effectuate presentation of governments and/or payers associated with multiple health care organizations for investment and reimbursement purposes. In some embodiments, responsive to a selection of a specific program by user 34, all of the involved stakeholders are highlighted on the map to show the connections with respect to one another. In some embodiments, the map facilitates visualization of the cash flow for one or both of the costs and savings. In some embodiments, presentation component 32 is configured to display different roles (e.g. payer, provider, commissioner, etc.) by changing the color of the icons (e.g., a color per category, taking into account that a stakeholder can have multiple roles in a program).

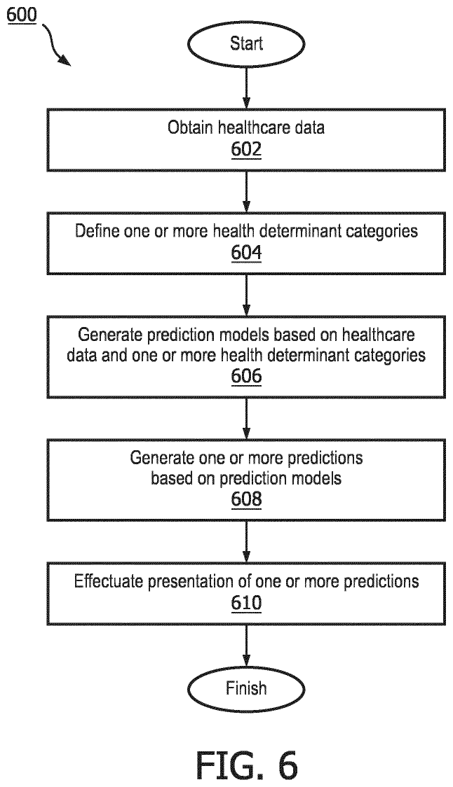

FIG. 6 illustrates a method 600 for providing prediction models for predicting a health determinant category contribution in clinical outcomes with a system. The system comprises one or more processors and/or other components. The one or more processors are configured by machine readable instructions to execute computer program components. The computer program components include a communications component, a model generation component, a prediction component, a presentation, and/or other components. The operations of method 600 presented below are intended to be illustrative. In some embodiments, method 600 may be accomplished with one or more additional operations not described, and/or without one or more of the operations discussed. Additionally, the order in which the operations of method 600 are illustrated in FIG. 6 and described below is not intended to be limiting.

In some embodiments, method 600 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 600 in response to instructions stored electronically on an electronic storage medium. The one or more processing devices may include one or more devices configured through hardware, firmware, and/or software to be specifically designed for execution of one or more of the operations of method 600.

At an operation 602, healthcare data is obtained. In some embodiments, the healthcare data includes (i) historical and financial data corresponding to one or more clinical programs, (ii) demographic, clinical, and behavioral data of one or more patients, and (iii) one or more environmental factors associated with the one or more clinical programs. In some embodiments, operation 602 is performed by a processor component the same as or similar to communications component 26 (shown in FIG. 1 and described herein).

At an operation 604, one or more health determinant categories are defined. In some embodiments, operation 604 is performed by a processor component the same as or similar to communications component 26 (shown in FIG. 1 and described herein).

At an operation 606, prediction models are generated based on the healthcare data and the one or more health determinant categories, such that, at least one of the prediction models is configured to generate a prediction related to a contribution of one or more constituents of the one or more health determinant categories to savings generated by the one or more clinical programs. In some embodiments, operation 606 is performed by a processor component the same as or similar to model generation component 28 (shown in FIG. 1 and described herein).

At an operation 608, one or more predictions are generated based on the prediction models. In some embodiments, the predictions are related to a contribution of the one or more health determinant categories to the savings generated by the one or more clinical programs. In some embodiments, operation 608 is performed by a processor component the same as or similar to prediction component 30 (shown in FIG. 1 and described herein).

At an operation 610, the one or more predictions are presented. In some embodiments, operation 610 is caused by a processor component the same as or similar to presentation component 32 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A system configured to provide prediction models for predicting a health determinant category contribution in savings generated by a clinical program, the system comprising:
one or more processors configured by machine-readable instructions to:
obtain healthcare data including (i) historical and financial data corresponding to one or more clinical programs, (ii) demographic, clinical, and behavioral data of one or more patients, and (iii) one or more environmental factors associated with the one or more clinical programs;
define one or more health determinant categories;
generate prediction models based on the healthcare data and the one or more health determinant categories such that at least one of the prediction models is configured to generate a prediction related to a contribution of one or more constituents of the one or more health determinant categories to savings generated by the one or more clinical programs;
generate one or more predictions based on the prediction models, the predictions being related to a contribution of the one or more health determinant categories to the savings generated by the one or more clinical programs; and
effectuate, via a user interface, presentation of the one or more predictions.

2. The system of claim 1, wherein the one or more processors are configured such that the contribution of one or more constituents of the one or more health determinant categories to the savings generated by the one or more clinical programs is predicted based on a Random forests model.

3. The system of claim 1, wherein the one or more health determinant categories includes one or more of patients' behavioral information, patients' clinical and demographic information, healthcare providers' information, environmental information, pre-post program behavioral information, pre-post program clinical information, pre-post program healthcare provider information, or pre-post environmental information.

4. The system of claim 3, further comprising one or more sensors configured to generate output signals conveying information related to geographical areas where the one or more patients spend their time, wherein the one or more processors are further configured to (i) merge, based on the output signals, one or both of the patients' behavioral information or the patients' clinical and demographic information with one or both of the healthcare providers' information or the environmental information and (ii) generate the prediction models based on the merged data.

5. The system of claim 3, wherein the one or more processors are further configured to determine, based on one or more of the pre-post program behavioral information, pre-post program clinical information, pre-post program healthcare provider information, or pre-post environmental information, a contribution of a change in one or more of the patients' behavioral information, the patients' clinical and demographic information, the healthcare providers' information, or the environmental information to the savings generated by the one or more clinical programs.

6. The system of claim 3, wherein the healthcare providers' information includes (i) a capitated payment received by a healthcare provider per patient and (ii) actual costs incurred by the healthcare provider to care for individual ones of the one or more patients, and wherein the one or more processors are further configured to determine cost savings by determining a difference between the capitated payment received by the healthcare provider per patient and the actual incurred costs by the healthcare provider to care for the individual ones of the one or more patients.

7. The system of claim 6, wherein the one or more processors are further configured to (i) determine a ratio between the determined cost savings and the actual incurred costs per the one or more health determinant categories and (ii) identify the most cost-effective health determinant category based on the determined ratio.

8. A method for providing prediction models for predicting a health determinant category contribution in savings generated by a clinical program with a system, the system comprising one or more processors configured by machine readable instructions, the method comprising:
obtaining, with the one or more processors, healthcare data including (i) historical and financial data corresponding to one or more clinical programs, (ii) demographic, clinical, and behavioral data of one or more patients, and (iii) one or more environmental factors associated with the one or more clinical programs;
defining, with the one or more processors, one or more health determinant categories;
generating, with the one or more processors, prediction models based on the healthcare data and the one or more health determinant categories such that at least one of the prediction models is configured to generate a prediction related to a contribution of one or more constituents of the one or more health determinant categories to savings generated by the one or more clinical programs;
generating, with the one or more processors, one or more predictions based on the prediction models, the predictions being related to a contribution of the one or more health determinant categories to the savings generated by the one or more clinical programs; and
effectuating, with a user interface, presentation of the one or more predictions.

9. The method of claim 8, wherein the contribution of one or more constituents of the one or more health determinant categories to the savings generated by the one or more clinical programs is predicted based on a Random forests model.

10. The method of claim 8, wherein the one or more health determinant categories includes one or more of patients' behavioral information, patients' clinical and demographic information, healthcare providers' information, environmental information, pre-post program behavioral information, pre-post program clinical information, pre-post program healthcare provider information, or pre-post environmental information.

11. The method of claim 10, wherein the system further comprises one or more sensors configured to generate output signals conveying information related to geographical areas where the one or more patients spend their time, wherein the method further comprises (i) merging, based on the output signals, one or both of the patients' behavioral information or the patients' clinical and demographic information with one or both of the healthcare providers' information or the environmental information and (ii) generating, with the one or more processors, the prediction models based on the merged data.

12. The method of claim 10, further comprising determining, based on one or more of the pre-post program behavioral information, pre-post program clinical information, pre-post program healthcare provider information, or pre-post environmental information, a contribution of a change in one or more of the patients' behavioral information, the patients' clinical and demographic information, the healthcare providers' information, or the environmental information to the savings generated by the one or more clinical programs.

13. The method of claim 10, wherein the healthcare providers' information includes (i) a capitated payment received by a healthcare provider per patient and (ii) actual costs incurred by the healthcare provider to care for individual ones of the one or more patients, and wherein the method further comprises determining, with the one or more processors, cost savings by determining a difference between the capitated payment received by the healthcare provider per patient and the actual incurred costs by the healthcare provider to care for the individual ones of the one or more patients.

14. The method of claim 13, further comprising (i) determining, with the one or more processors, a ratio between the determined cost savings and the actual incurred costs per the one or more health determinant categories and (ii) identifying, with the one or more processors, the most cost-effective health determinant category based on the determined ratio.

15. A system for providing prediction models for predicting a health determinant category contribution in savings generated by a clinical program, the system comprising:
means for obtaining healthcare data including (i) historical and financial data corresponding to one or more clinical programs, (ii) demographic, clinical, and behavioral data of one or more patients, and (iii) one or more environmental factors associated with the one or more clinical programs;
means for defining one or more health determinant categories;
means for generating prediction models based on the healthcare data and the one or more health determinant categories such that at least one of the prediction models is configured to generate a prediction related to a contribution of one or more constituents of the one or more health determinant categories to savings generated by the one or more clinical programs;
means for generating one or more predictions based on the prediction models, the predictions being related to a contribution of the one or more health determinant categories to the savings generated by the one or more clinical programs; and
means for effectuating presentation of the one or more predictions.

16. The system of claim 15, wherein the contribution of one or more constituents of the one or more health determinant categories to the savings generated by the one or more clinical programs is predicted based on a Random forests model.

17. The system of claim 15, wherein the one or more health determinant categories includes one or more of patients' behavioral information, patients' clinical and demographic information, healthcare providers' information, environmental information, pre-post program behavioral information, pre-post program clinical information, pre-post program healthcare provider information, or pre-post environmental information.

18. The system of claim 17, further comprising:
means for generating output signals conveying information related to geographical areas where the one or more patients spend their time;
means for merging, based on the output signals, one or both of the patients' behavioral information or the patients' clinical and demographic information with one or both of the healthcare providers' information or the environmental information; and
means for generating the prediction models based on the merged data.

19. The system of claim 17, further comprising means for determining, based on one or more of the pre-post program behavioral information, pre-post program clinical information, pre-post program healthcare provider information, or pre-post environmental information, a contribution of a change in one or more of the patients' behavioral information, the patients' clinical and demographic information, the healthcare providers' information, or the environmental information to the savings generated by the one or more clinical programs.

20. The method of claim 17, wherein the healthcare providers' information includes (i) a capitated payment received by a healthcare provider per patient and (ii) actual costs incurred by the healthcare provider to care for individual ones of the one or more patients, and wherein the system further comprises:
means for determining cost savings by determining a difference between the capitated payment received by the healthcare provider per patient and the actual incurred costs by the healthcare provider to care for the individual ones of the one or more patients;
means for determining a ratio between the determined cost savings and the actual incurred costs per the one or more health determinant categories; and
means for identifying the most cost-effective health determinant category based on the determined ratio.
